(19) United States
(12) Patent Application Publication
(10) Pub. No.: US 2020/0147175 A1
(43) Pub. Date: May 14, 2020

(54) METHOD FOR TREATING ANTI-HER2 THERAPY-RESISTANT MUC4+ HER2+ CANCER

(71) Applicant: CONSEJO NACIONAL DE INVESTIGACIONES CIENTÍFICAS Y, BUENOS AIRES (AR)

(72) Inventor: ROXANA SCHILLACI, BUENOS AIRES (AR)

(73) Assignee: CONSEJO NACIONAL DE INVESTIGACIONES CIENTÍFICAS Y TÉCNICAS, BUENOS AIRES (AR)

(21) Appl. No.: 16/679,272

(22) Filed: Nov. 10, 2019

Related U.S. Application Data
(60) Provisional application No. 62/758,575, filed on Nov. 10, 2018.

(51) Int. Cl. A61K 38/19; G01N 33/574; A61K 39/395; A61K 45/06; A61P 35/00; A61K 35/28

(52) U.S. Cl. CPC A61K 38/191; G01N 33/57492; A61K 39/39558; A61K 2039/545; A61P 35/00; A61K 35/28; A61K 45/06

(57) ABSTRACT

A method for treating a patient suffering from an anti-HER2-resistant, MUC4+ HER2+ cancer is disclosed. For example, the method can be used to treat trastuzumab-resistant HER2+ breast cancer. In some embodiments, the method includes: (i) determining mucin-4 (MUC4) expression in cells of the patient; and (ii) if the MUC4 expression is greater than a predetermined-threshold, administering to the patient a therapeutically effective amount of a selective inhibitor of soluble tumor necrosis factor in combination with an anti-HER2 therapeutic agent, whereby said patient is treated.

### TECHNICAL FIELD

The invention relates generally to the administration of a selective inhibitor of soluble tumor necrosis factor alpha (TNF-α), preferably a dominant negative TNF-α protein, in combination with an anti-HER2 therapeutic agent for treating HER2-positive breast cancer.

### BACKGROUND ART

HER2/neu overexpression/amplification occurs in approximately twenty percent of invasive breast cancers and is strongly associated with poor prognosis. Trastuzumab, the first clinically approved monoclonal antibody (mAb) against HER2, is the standard-of-care treatment for patients with early and metastatic HER2-positive breast cancer, administered alone or in combination with chemotherapy. Trastuzumab's mechanism of action relies mainly on inhibiting HER2-mediated signal transduction and triggering antibody-dependent cell-mediated cytotoxicity (ADCC). The administration of trastuzumab profoundly improved the outcome of HER2-positive breast cancer patients. However, up to forty-two percent of the patients treated with neoadjuvant trastuzumab and twenty-seven percent of those with adjuvant trastuzumab experience disease progression. The lack of sustained response can be due to de novo or acquired resistance. Multiple mechanisms underlying trastuzumab resistance in breast cancer have been described, including persistent activation of the PI3K-Akt pathway, cross-talk of heterologous receptor signaling pathways and cleavage of HER2 extracellular domain, among others.

Mucin 4 (MUC4) is a glycoprotein that belongs to the membrane-bound family of mucins and has two non-covalently associated subunits encoded by a single gene. The extracellular subunit, MUC4a, is heavily glycosylated and provides anti-adhesive properties to the cell. The transmembrane subunit MUC4β, contains two EGF-like domains in the extracellular portion that can interact with HER2. MUC4 is normally expressed in the apical region of mammary cells. In breast cancer cells, it is aberrantly expressed in the cytosol and shows an increased expression pattern in metastatic lesions, compared to primary-matched tumors. In addition, MUC4 has been reported to mask the trastuzumab-binding epitope of HER2, thereby decreasing its binding in vitro in JIMT-1, a de novo trastuzumab-resistant breast cancer cell line.

TNF-α is a 17-kDa pro-inflammatory cytokine that is produced by macrophages and T-lymphocytes after bacterial endotoxin exposure. In addition, TNF-α can be produced by cancer cells. TNF-α is a structural glycoprotein with a transmembrane region. By the action of proteases (such as ADAM17/TACE) transmembrane TNF-α (tmTNF) become soluble. Either tmTNF or soluble TNF are structurally a homotrimer.

The presence of inflammatory mediators in the tumor microenvironment, either generated by the tumor cells or by tumor-infiltrating cells, is widely recognized in cancer biology. In particular, chronic inflammation plays a key role in the pathogenesis and progression of breast cancer. Indeed, inflammation has been associated with poor prognosis in breast cancer patients and with an increased risk of recurrence. Moreover, NF-kB signaling, the main transcription factor induced by inflammatory mediators, such as cytokines, is activated in cells resistant to anti-estrogen therapies.

There is an immediate need for improved therapeutic strategies targeting HER2-positive breast cancer in patients that are at risk for trastuzumab resistance or those that are trastuzumab-resistant.

### SUMMARY OF INVENTION

It has been discovered that soluble TNF-α turns trastuzumab-sensitive cells and tumors into resistant ones by upregulating MUC4 expression, which reduces trastuzumab binding to its epitope and impairs antibody-dependent cell-mediated cytotoxicity (ADCC).

It has been identified that MUC4 expression, in HER2-positive breast cancer samples, is an independent predictor of poor disease-free survival in patients treated with adjuvant trastuzumab.

It has been further discovered that treatment with TNFα inhibitors, specifically XPRO1595, a dominant negative of soluble TNF-α (solTNF) protein, in combination with trastuzumab, pertuzumab, or a combination thereof, resulted in a significant decrease in JIMT-1 cell proliferation compared to immunoglobulin G (IgG), monotherapies of trastuzumab and pertuzumab, and a combination of trastuzumab and pertuzumab. JIMT-1 cells are de novo resistant to trastuzumab and pertuzumab. These results suggest that a TNFα blockade sensitizes JIMT-1 cells resistant to trastuzumab and pertuzumab treatment.

It was identified that the combination of trastuzumab, pertuzumab, and a TNF-inhibitor, specifically XPRO1595, caused a significant decrease in JIMT-1 tumor volume. Meanwhile, tumors were unresponsive to trastuzumab plus pertuzumab, and pertuzumab plus XPRO1595 treatments.

The effect of trastuzumab plus XPRO1595 was found to be indistinguishable from that of trastuzumab plus pertuzumab and XPRO1595, indicating that pertuzumab is not contributing to the antiproliferative effect of trastuzumab plus XPRO1595 in the JIMT-1 experimental model.

It was demonstrated that XPRO1595 was able to decrease MUC4 expression in JIMT-1 tumor when administered in vivo, suggesting that solTNF is responsible for MUC4 regulation.

In accordance with these findings, herein disclosed is a method for treating a HER2-positive breast cancer patient, in particular one that is resistant to trastuzumab, the method comprising: (i) determining mucin-4 (MUC4) expression in cells of the patient; and (ii) if the MUC4 expression is greater than a predetermined-threshold, administering to the patient a therapeutically effective amount of a selective inhibitor of solTNF in combination with an anti-HER2 therapeutic agent, whereby said patient is treated. This method may be optionally extended to patients with early or metastatic HER2 positive breast cancer at diagnosis, to avoid resistance.

Other particulars and variations are described in the detailed description and the drawings appended hereto.

### BRIEF DESCRIPTION OF DRAWINGS

FIG. 1A shows the nucleic acid sequence of human TNF-α (SEQ ID NO:1). An additional six histidine codons, located between the start codon and the first amino acid, are underlined.

FIG. 1B shows the amino acid sequence of human TNF-α (SEQ ID NO:2) with an additional 6 histidines (underlined) between the start codon and the first amino acid. Amino acids changed in exemplary TNF-α variants are shown in bold.

FIG. 1C shows the amino acid sequence of human TNF-α (SEQ ID NO:3).

FIG. 2 shows the positions and amino acid changes in certain TNF-α variants.

FIG. 3 shows cell proliferation in JIMT-1 cell line with treatment of various therapeutic components.

FIG. 4A shows a plot of tumoral volume vs. days of treatment for a variety of treatment regimes.

FIG. 4B shows a chart of tumor growth rate, volume, and percent inhibition with respect to a variety of treatment regimens applied to JIMT-1 tumor.

FIG. 4C shows MUC4 expression performed in protein extracts of a variety of treatment regimens applied to JIMT-1 tumor.

FIG. 5 shows pathology scoring of MUC4 in breast cancer biopsies by immunohistochemistry.

FIG. 6 shows Kaplan-Meier analysis of the probability of disease-free survival of patients with HER2-positive tumors who received adjuvant trastuzumab treatment based on the expression of MUC4.

FIG. 7A shows forest plots indicating the hazard ratios (squares) and 95% confidence interval (horizontal lines) of univariate subgroup analysis.

FIG. 7B shows forest plots indicating the hazard ratios (squares) and 95% confidence interval (horizontal lines) of multivariate subgroup analysis.

FIG. 8A is a bar chart illustrating migration of JIMT-1 cells according to scratch assay.

FIG. 8B is a bar chart illustrating migration of JIMT-1 cells according to invasion assay.

FIG. 9A is a bar chart illustrating MUC4 silencing by doxycycline treatment sensitizes JIMT-1 cells to trastuzumab and that addition of XPRO1595 did not further induce a decrease in cell proliferation.

FIG. 9B is a bar chart illustrating JIMT-1-shControl with doxycycline decreased their proliferation only when trastuzumab and XPRO1595 were present.

FIG. 10A is a bar chart illustrating knockdown of MUC4 expression revealed that trastuzumab treatment was effective in inhibiting JIMT-1-shMUC4 tumor growth at comparable levels to trastuzumab+XPRO1595 administration.

FIG. 10B is a bar chart illustrating that only trastuzumab+XPRO1595 administration was able to inhibit tumor growth in control groups.

FIG. 10C is a bar chart illustrating JIMT-1-sh-Control tumors with doxycycline administration behaved like JIMT-1 wildtype tumors.

FIG. 10D is a bar chart illustrating JIMT-1-sh-Control tumors without doxycycline administration also behaved like JIMT-1 wildtype tumors.

FIG. 11A is a bar chart illustrating that tumor growth inhibition was accompanied by an increase in NK cells activation.

FIG. 11B is a bar chart illustrating that tumor growth inhibition was accompanied by an increase in NK cells degranulation.

FIG. 12A is a bar chart illustrating that tumor growth inhibition was accompanied by a decrease in the recruitment of myeloid cells vs. IgG.

FIG. 12B is a bar chart illustrating Trastuzumab and trastuzumab+XPRO1595 increases G-MDSC vs IgG with doxycycline.

FIG. 12C is a bar chart illustrating trastuzumab+XPRO1595 sharply decreases M-MDSC presence in JIMT-shMUC4 tumors without doxycycline vs IgG.

FIG. 13A is a bar chart illustrating macrophages influx to the tumor bed was stimulated by the absence of MUC4 and was further increased in tumors treated with trastuzumab or trastuzumab+XPRO1595 vs IgG.

FIG. 13B is a bar chart illustrating when MUC4 was silenced, an increase in M1/M2 macrophages ratio was observed in the trastuzumab and trastuzumab+XPRO1595 treated groups vs IgG group.

FIG. 14A shows representative cases of HER2+/MUC4+ tumors that lack TILs and HER2+/MUC4− tumors that had abundant TILs.

FIG. 14B shows an inverse correlation between MUC4 expression and TILs presence.

### DESCRIPTION OF EMBODIMENTS

Disclosed herein is the novel and unexpected finding that inhibition of soluble TNF-α can be used as a strategy to reduce (downregulate) or prevent upregulation of MUC4 on the surface of HER2-positive breast cancer cells, which in turn enhances binding of trastuzumab and other anti-HER2 therapeutic agents for effectuating the treatment of HER2 positive breast cancer in trastuzumab-resistant and other anti-HER2 treatment-resistive cells.

### Selective Inhibitors of Soluble Tumor Necrosis Factor

Proteins with TNF-α antagonist activity, and nucleic acids encoding these proteins, were previously discovered which function to inhibit the soluble form of TNF-α (solTNF) without inhibiting transmembrane TNF-α (tmTNF); collectively these proteins and nucleic acids encoding these proteins are herein collectively referred to as “selective inhibitors of solTNF”.

Examples of selective inhibitors of solTNF are disclosed in U.S. Pat. Nos. 7,056,695; 7,101,974; 7,144,987; 7,244,823; 7,446,174; 7,662,367; and 7,687,461; the entire contents of each of which is hereby incorporated by reference.

Preferred selective inhibitors of solTNF may be dominant negative TNFα proteins, referred to herein as “DNTNF-α,” “DN-TNF-α proteins,” “TNFα variants,” “TNFα variant proteins,” “variant TNF-α,” “variant TNF-α,” and the like. By “variant TNF-α” or “TNF-α proteins” is meant TNFα or TNF-α proteins that differ from the corresponding wild type protein by at least 1 amino acid. Thus, a variant of human TNF-α is compared to SEQ ID NO:1 (nucleic acid including codons for 6 histidines), SEQ ID NO:2 (amino acid including 6 N-terminal histidines) or SEQ ID NO:3 (amino acid without 6 N-terminal histidines). DN-TNF-α proteins are disclosed in detail in U.S. Pat. No. 7,446,174, which is incorporated herein in its entirety by reference. As used herein variant TNF-α or TNF-α proteins include TNF-α monomers, dimers or trimers. Included within the definition of “variant TNF-α” are competitive inhibitor TNF-α variants. While certain variants as described herein, one of skill in the art will understand that other variants may be made while retaining the function of inhibiting soluble but not transmembrane TNF-α.

Thus, the proteins of the invention are antagonists of wild type TNF-α. By “antagonists of wild type TNF-α” is meant that the variant TNF-α protein inhibits or significantly decreases at least one biological activity of wild-type TNF-α.

In a preferred embodiment the variant is antagonist of soluble TNF-α, but does not significantly antagonize transmembrane TNF-α, e.g., DN-TNF-α protein as disclosed herein inhibits signaling by soluble TNF-α, but not transmembrane TNF-α. By “inhibits the activity of TNF-α” and grammatical equivalents is meant at least a 10% reduction in wild-type, soluble TNF-α, more preferably at least a 50% reduction in wild-type, soluble TNF-α activity, and even more preferably, at least 90% reduction in wild-type, soluble TNF-α activity. Preferably there is an inhibition in wild-type soluble TNF-α activity in the absence of reduced signaling by transmembrane TNF-α. In a preferred embodiment, the activity of soluble TNF-α is inhibited while the activity of transmembrane TNF-α is substantially and preferably completely maintained.

The TNF proteins useful in various embodiments of the invention have modulated activity as compared to wild type proteins. In a preferred embodiment, variant TNF-α proteins exhibit decreased biological activity (e.g. antagonism) as compared to wild type TNF-α, including but not limited to, decreased binding to a receptor (p55, p75 or both), decreased activation and/or ultimately a loss of cytotoxic activity. By “cytotoxic activity” herein refers to the ability of a TNF-α variant to selectively kill or inhibit cells. Variant TNF-α proteins that exhibit less than 50% biological activity as compared to wild type are preferred. More preferred are variant TNF-α proteins that exhibit less than 25%, even more preferred are variant proteins that exhibit less than 15%, and most preferred are variant TNF-α proteins that exhibit less than 10% of a biological activity of wild-type TNF-α. Suitable assays include, but are not limited to, caspase assays, TNF-α cytotoxicity assays, DNA binding assays, transcription assays (using reporter constructs), size exclusion chromatography assays and radiolabeling/immuno-precipitation,), and stability assays (including the use of circular dichroism (CD) assays and equilibrium studies), according to methods know in the art.

In one embodiment, at least one property critical for binding affinity of the variant TNF-α proteins is altered when compared to the same property of wild type TNF-α and in particular, variant TNF-α proteins with altered receptor affinity are preferred. Particularly preferred are variant TNF-α with altered affinity toward oligomerization to wild type TNF-α. Thus, the invention makes use of variant TNF-α proteins with altered binding affinities such that the variant TNF-α proteins will preferentially oligomerize with wild type TNF-α, but do not substantially interact with wild type TNF receptors, i.e., p55, p75. “Preferentially” in this case means that given equal amounts of variant TNF-α monomers and wild type TNF-α monomers, at least 25% of the resulting trimers are mixed trimers of variant and wild type TNF-α, with at least about 50% being preferred, and at least about 80-90% being particularly preferred. In other words, it is preferable that the variant TNF-α proteins implemented in embodiments of the invention have greater affinity for wild type TNF-α protein as compared to wild type TNF-α proteins. By “do not substantially interact with TNF receptors” is meant that the variant TNF-α proteins will not be able to associate with either the p55 or p75 receptors to significantly activate the receptor and initiate the TNF signaling pathway(s). In a preferred embodiment, at least a 50% decrease in receptor activation is seen, with greater than 50%, 75%, 80-90% being preferred.

In some embodiments, the variants of the invention are antagonists of both soluble and transmembrane TNF-α. However, as described herein, preferred variant TNF-α proteins are antagonists of the activity of soluble TNF-α but do not substantially affect the activity of transmembrane TNF-α. Thus, a reduction of activity of the heterotrimers for soluble TNF-α is as outlined above, with reductions in biological activity of at least 10%, 25, 50, 75, 80, 90, 95, 99 or 100% all being preferred. However, some of the variants outlined herein comprise selective inhibition; that is, they inhibit soluble TNF-α activity but do not substantially inhibit transmembrane TNF-α. In these embodiments, it is preferred that at least 80%, 85, 90, 95, 98, 99 or 100% of the transmembrane TNF-α activity is maintained. This may also be expressed as a ratio; that is, selective inhibition can include a ratio of inhibition of soluble to transmembrane TNF-α. For example, variants that result in at least a 10:1 selective inhibition of soluble to transmembrane TNF-α activity are preferred, with 50:1, 100:1, 200:1, 500:1, 1000:1 or higher find particular use in the invention. Thus, one embodiment utilizes variants, such as double mutants at positions 87/145 as outlined herein, that substantially inhibit or eliminate soluble TNF-α activity (for example by exchanging with homotrimeric wild-type to form heterotrimers that do not bind to TNF-α receptors or that bind but do not activate receptor signaling) but do not significantly affect (and preferably do not alter at all) transmembrane TNF-α activity. Without being bound by theory, the variants exhibiting such differential inhibition allow the decrease of inflammation without a corresponding loss in immune response.

In one embodiment, the affected biological activity of the variants is the activation of receptor signaling by wild type TNF-α proteins. In a preferred embodiment, the variant TNF-α protein interacts with the wild type TNF-α protein such that the complex comprising the variant TNF-α and wild type TNF-α has reduced capacity to activate (as outlined above for “substantial inhibition”), and in preferred embodiments is incapable of activating, one or both of the TNF receptors, i.e. p55 TNF-R or p75 TNF-R. In a preferred embodiment, the variant TNF-α protein is a variant TNF-α protein that functions as an antagonist of wild type TNF-α. Preferably, the variant TNF-α protein preferentially interacts with wild type TNF-α to form mixed trimers with the wild type protein such that receptor binding does not significantly occur and/or TNF-α signaling is not initiated. By mixed trimers is meant that monomers of wild type and variant TNF-α proteins interact to form heterotrimeric TNF-α. Mixed trimers may comprise 1 variant TNF-α protein:2 wild type TNF-α proteins, 2 variant TNF-α proteins:1 wild type TNF-α protein. In some embodiments, trimers may be formed comprising only variant TNF-α proteins.

The variant TNF-α antagonist proteins implemented in embodiments of the invention are highly specific for TNF-α antagonism relative to TNF-beta antagonism. Additional characteristics include improved stability, pharmacokinetics, and high affinity for wild type TNF-α. Variants with higher affinity toward wild type TNF-α may be generated from variants exhibiting TNF-α antagonism as outlined above.

Similarly, variant TNF-α proteins, for example are experimentally tested and validated in in vivo and in in vitro assays. Suitable assays include, but are not limited to, activity assays and binding assays. For example, TNF-α activity assays, such as detecting apoptosis via caspase activity can be used to screen for TNF-α variants that are antagonists of wild type TNF-α. Other assays include using the Sytox green nucleic acid stain to detect TNF-induced cell permeability in an Actinomycin-D sensitized cell line. As this stain is excluded from live cells, but penetrates dying cells, this assay also can be used to detect TNF-α variants that are agonists of wild-type TNF-α. By “agonists of wild type TNF-α” is meant that the variant TNF-α protein enhances the activation of receptor signaling by wild type TNF-α proteins. Generally, variant TNF-α proteins that function as agonists of wild type TNF-α are not preferred. However, in some embodiments, variant TNF-α proteins that function as agonists of wild type TNF-α protein are preferred. An example of an NF kappaB assay is presented in Example 7 of U.S. Pat. No. 7,446,174, which is expressly incorporated herein by reference.

In a preferred embodiment, binding affinities of variant TNF-α proteins as compared to wild type TNF-α proteins for naturally occurring TNF-α and TNF receptor proteins such as p55 and p75 are determined. Suitable assays include, but are not limited to, e.g., quantitative comparisons comparing kinetic and equilibrium binding constants, as are known in the art. Examples of binding assays are described in Example 6 of U.S. Pat. No. 7,446,174, which is expressly incorporated herein by reference.

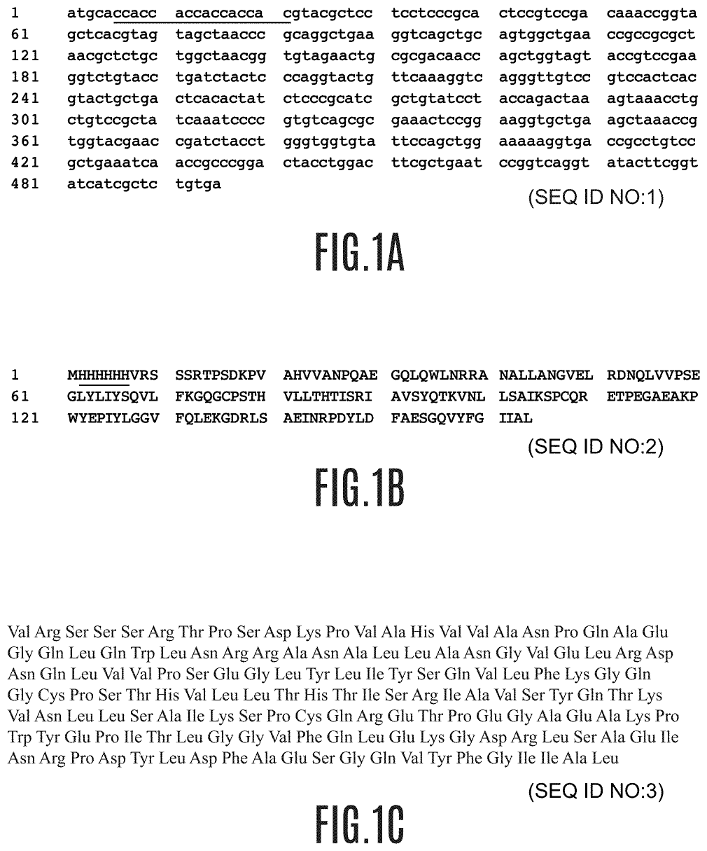

In a preferred embodiment, the variant TNF-α protein has an amino acid sequence that differs from a wild type TNF-α sequence by at least 1 amino acid, with from 1, 2, 3, 4, 5, 6, 7, 8, 9 and 10 amino acids all contemplated, or higher. Expressed as a percentage, the variant TNF-α proteins of the invention preferably are greater than 90% identical to wild-type, with greater than 95, 97, 98 and 99% all being contemplated. Stated differently, based on the human TNF-α sequence of FIG. 1B (SEQ ID NO:2) excluding the N-terminal 6 histidines, as shown in FIG. 1C (SEQ ID NO:3), variant TNF-α proteins have at least about 1 residue that differs from the human TNF-α sequence, with at least about 2, 3, 4, 5, 6, 7 or 8 different residues. Preferred variant TNF-α proteins have 3 to 8 different residues.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). In a similar manner, “percent (%) nucleic acid sequence identity” with respect to the coding sequence of the polypeptides identified is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

TNF-α proteins may be fused to, for example, other therapeutic proteins or to other proteins such as Fc or serum albumin for therapeutic or pharmacokinetic purposes. In this embodiment, a TNF-α protein implemented in embodiments of the invention is operably linked to a fusion partner. The fusion partner may be any moiety that provides an intended therapeutic or pharmacokinetic effect. Examples of fusion partners include but are not limited to Human Serum Albumin, a therapeutic agent, a cytotoxic or cytotoxic molecule, radionucleotide, and an Fc, etc. As used herein, an Fc fusion is synonymous with the terms “immunoadhesin”, “Ig fusion”, “Ig chimera”, and “receptor globulin” as used in the prior art (Chamow et al., 1996, Trends Biotechnol 14:52-60; Ashkenazi et al., 1997, CurrOpin Immunol 9:195-200, both incorporated by reference). An Fc fusion combines the Fc region of an immunoglobulin with the target-binding region of a TNF-α protein, for example. See for example U.S. Pat. Nos. 5,766,883 and 5,876,969, both of which are hereby incorporated by reference.

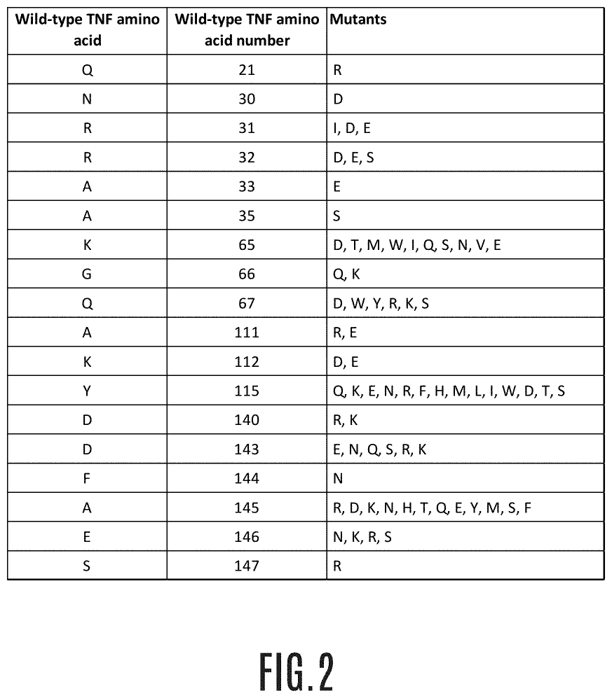

In a preferred embodiment, the variant TNF-α proteins comprise variant residues selected from the following positions 21, 23, 30, 31, 32, 33, 34, 35, 57, 65, 66, 67, 69, 75, 84, 86, 87, 91, 97, 101, 111, 112, 115, 140, 143, 144, 145, 146, and 147. Preferred amino acids for each position, including the human TNF-α residues, are shown in FIG. 2. Thus, for example, at position 143, preferred amino acids are Glu, Asn, Gln, Ser, Arg, and Lys; etc. Preferred changes include: V1M, Q21C, Q21R, E23C, R31C, N34E, V91E, Q21R, N30D, R31C, R311, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, I97R, I97T, C101A, A111R, A111E, K112D, K112E, Y115D, Y115E, Y115F, Y115H, Y115I, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R. These may be done either individually or in combination, with any combination being possible. However, as outlined herein, preferred embodiments utilize at least 1 to 8, and preferably more, positions in each variant TNF-α protein.

In an additional aspect, the invention provides TNF-α variants selected from the group consisting of: XENP268 XENP344, XENP345, XENP346, XENP550, XENP551, XENP557, XENP1593, XENP1594, and XENP1595 as outlined in Example 3 OF U.S. Pat. No. 7,662,367, which is incorporated herein by reference.

In an additional aspect, the invention makes use of methods of forming a TNF-α heterotrimer in vivo in a mammal comprising administering to the mammal a variant TNF-α molecule as compared to the corresponding wild-type mammalian TNF-α, wherein said TNF-α variant is substantially free of agonistic activity.

In an additional aspect, the invention makes use of methods of screening for selective inhibitors comprising contacting a candidate agent with a soluble TNF-α protein and assaying for TNF-α biological activity; contacting a candidate agent with a transmembrane TNF-α protein and assaying for TNF-α biological activity, and determining whether the agent is a selective inhibitor. The agent may be a protein (including peptides and antibodies, as described herein) or small molecules.

In a further aspect, the invention makes use of variant TNF-α proteins that interact with the wild type TNF-α to form mixed trimers incapable of activating receptor signaling. Preferably, variant TNF-α proteins with 1, 2, 3, 4, 5, 6 and 7 amino acid changes are used as compared to wild type TNF-α protein. In a preferred embodiment, these changes are selected from positions 1, 21, 23, 30, 31, 32, 33, 34, 35, 57, 65, 66, 67, 69, 75, 84, 86, 87, 91, 97, 101, 111, 112, 115, 140, 143, 144, 145, 146 and 147. In an additional aspect, the non-naturally occurring variant TNF-α proteins have substitutions selected from the group of substitutions consisting of: V1M, Q21C, Q21R, E23C, N34E, V91E, Q21R, N30D, R31C, R311, R31D, R31E, R32D, R32E, R32S, A33E, N34E, N34V, A35S, D45C, L57F, L57W, L57Y, K65D, K65E, K651, K65M, K65N, K65Q, K65T, K65S, K65V, K65W, G66K, G66Q, Q67D, Q67K, Q67R, Q67S, Q67W, Q67Y, C69V, L75E, L75K, L75Q, A84V, S86Q, S86R, Y87H, Y87R, V91E, I97R, I97T, C101A, A111R, A111E, K112D, K112E, Y115D, Y115E, Y115F, Y115H, Y115I, Y115K, Y115L, Y115M, Y115N, Y115Q, Y115R, Y115S, Y115T, Y115W, D140K, D140R, D143E, D143K, D143L, D143R, D143N, D143Q, D143R, D143S, F144N, A145D, A145E, A145F, A145H, A145K, A145M, A145N, A145Q, A145R, A145S, A145T, A145Y, E146K, E146L, E146M, E146N, E146R, E146S and S147R.

In another preferred embodiment, substitutions may be made either individually or in combination, with any combination being possible. Preferred embodiments utilize at least one, and preferably more, positions in each variant TNF-α protein. For example, substitutions at positions 31, 57, 69, 75, 86, 87, 97, 101, 115, 143, 145, and 146 may be combined to form double variants. In addition, triple, quadruple, quintuple and the like, point variants may be generated.

In one aspect the invention makes use of TNF-α variants comprising the amino acid substitutions A145R/197T. In one aspect, the invention provides TNF-α variants comprising the amino acid substitutions V1M, R31C, C69V, Y87H, C101A, and A145R. In a preferred embodiment, this variant is PEGylated.

In a preferred embodiment the variant is XPRO1595, a PEGylated protein comprising V1M, R31C, C69V, Y87H, C101A, and A145R mutations relative to the wild type human sequence, also referred to herein as “XPro”.

For purposes of the present invention, the areas of the wild type or naturally occurring TNF-α molecule to be modified are selected from the group consisting of the Large Domain (also known as II), Small Domain (also known as I), the DE loop, and the trimer interface. The Large Domain, the Small Domain and the DE loop are the receptor interaction domains. The modifications may be made solely in one of these areas or in any combination of these areas. The Large Domain preferred positions to be varied include: 21, 30, 31, 32, 33, 35, 65, 66, 67, 111, 112, 115, 140, 143, 144, 145, 146 and/or 147. For the Small Domain, the preferred positions to be modified are 75 and/or 97. For the DE Loop, the preferred position modifications are 84, 86, 87 and/or 91. The Trimer Interface has preferred double variants including positions 34 and 91 as well as at position 57. In a preferred embodiment, substitutions at multiple receptor interaction and/or trimerization domains may be combined. Examples include, but are not limited to, simultaneous substitution of amino acids at the large and small domains (e.g. A145R and I97T), large domain and DE loop (A145R and Y87H), and large domain and trimerization domain (A145R and L57F). Additional examples include any and all combinations, e.g., I97T and Y87H (small domain and DE loop). More specifically, theses variants may be in the form of single point variants, for example K112D, Y115K, Y115I, Y115T, A145E or A145R. These single point variants may be combined, for example, Y115I and A145E, or Y115I and A145R, or Y115T and A145R or Y115I and A145E; or any other combination.

Preferred double point variant positions include 57, 75, 86, 87, 97, 115, 143, 145, and 146; in any combination. In addition, double point variants may be generated including L57F and one of Y115I, Y115Q, Y115T, D143K, D143R, D143E, A145E, A145R, E146K or E146R. Other preferred double variants are Y115Q and at least one of D143N, D143Q, A145K, A145R, or E146K; Y115M and at least one of D143N, D143Q, A145K, A145R or E146K; and L57F and at least one of A145E or 146R; K65D and either D143K or D143R, K65E and either D143K or D143R, Y115Q and any of L75Q, L57W, L57Y, L57F, I97R, I97T, S86Q, D143N, E146K, A145R and I97T, A145R and either Y87R or Y87H; N34E and V91E; L75E and Y115Q; L75Q and Y115Q; L75E and A145R; and L75Q and A145R.

Further, triple point variants may be generated. Preferred positions include 34, 75, 87, 91, 115, 143, 145 and 146. Examples of triple point variants include V91 E, N34E and one of Y115I, Y115T, D143K, D143R, A145R, A145E E146K, and E146R. Other triple point variants include L75E and Y87H and at least one of Y115Q, A145R, Also, L75K, Y87H and Y115Q. More preferred are the triple point variants V91E, N34E and either A145R or A145E.

Variant TNF-α proteins may also be identified as being encoded by variant TNF-α nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred. In a preferred embodiment, a variant TNF-α nucleic acid encodes a variant TNF-α protein. As will be appreciated by those in the art, due to the degeneracy of the genetic code, an extremely large number of nucleic acids may be made, all of which encode the variant TNF-α proteins of the present invention. Thus, having identified a particular amino acid sequence, those skilled in the art could make any number of different nucleic acids, by simply modifying the sequence of one or more codons in a way which does not change the amino acid sequence of the variant TNF-α.

In one embodiment, the nucleic acid homology is determined through hybridization studies. Thus, for example, nucleic acids which hybridize under high stringency to the nucleic acid sequence shown in FIG. 1A (SEQ ID NO:1) or its complement and encode a variant TNF-α protein is considered a variant TNF-α gene. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993), incorporated by reference. Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. In another embodiment, less stringent hybridization conditions are used; for example, moderate or low stringency conditions may be used, as are known in the art; see Maniatis and Ausubel, supra, and Tijssen, supra. In addition, nucleic acid variants encode TNF-α protein variants comprising the amino acid substitutions described herein. In one embodiment, the TNF-α variant encodes a polypeptide variant comprising the amino acid substitutions A145R/197T. In one aspect, the nucleic acid variant encodes a polypeptide comprising the amino acid substitutions V1M, R31C, C69V, Y87H, C101A, and A145R, or any 1, 2, 3, 4 or 5 of these variant amino acids.

The variant TNF-α proteins and nucleic acids of the present invention are recombinant. As used herein, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments. The nucleic acid may be double stranded, single stranded, or contain portions of both double stranded or single stranded sequence. As will be appreciated by those in the art, the depiction of a single strand (“Watson”) also defines the sequence of the other strand (“Crick”); thus, the sequence depicted in FIG. 1A (SEQ ID NO:1) also includes the complement of the sequence. By the term “recombinant nucleic acid” is meant nucleic acid, originally formed in vitro, in general, by the manipulation of nucleic acid by endonucleases, in a form not normally found in nature. Thus, an isolated variant TNF-α nucleic acid, in a linear form, or an expression vector formed in vitro by ligating DNA molecules that are not normally joined, are both considered recombinant for the purposes of this invention.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild-type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a variant TNF-α protein from one organism in a different organism or host cell. Alternatively, the protein may be made at a significantly higher concentration than is normally seen, through the use of an inducible promoter or high expression promoter, such that the protein is made at increased concentration levels. Furthermore, all of the variant TNF-α proteins outlined herein are in a form not normally found in nature, as they contain amino acid substitutions, insertions and deletions, with substitutions being preferred, as discussed below.

Also included within the definition of variant TNF-α proteins of the present invention are amino acid sequence variants of the variant TNF-α sequences outlined herein and shown in the Figures. That is, the variant TNF-α proteins may contain additional variable positions as compared to human TNF-α. These variants fall into one or more of three classes: substitutional, insertional or deletional variants.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Using the nucleic acids disclosed herein, which encode a variant TNF-α protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the variant TNF-α protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

In a preferred embodiment, when the endogenous secretory sequence leads to a low level of secretion of the naturally occurring protein or of the variant TNF-α protein, a replacement of the naturally occurring secretory leader sequence is desired. In this embodiment, an unrelated secretory leader sequence is operably linked to a variant TNF-α encoding nucleic acid leading to increased protein secretion. Thus, any secretory leader sequence resulting in enhanced secretion of the variant TNF-α protein, when compared to the secretion of TNF-α and its secretory sequence, is desired. Suitable secretory leader sequences that lead to the secretion of a protein are known in the art. In another preferred embodiment, a secretory leader sequence of a naturally occurring protein or a protein is removed by techniques known in the art and subsequent expression results in intracellular accumulation of the recombinant protein.

Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the fusion protein; for example, transcriptional and translational regulatory nucleic acid sequences from Bacillus are preferably used to express the fusion protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In a preferred embodiment, the promoters are strong promoters, allowing high expression in cells, particularly mammalian cells, such as the CMV promoter, particularly in combination with a Tet regulatory element.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences that flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used. A preferred expression vector system is a retroviral vector system such as is generally described in PCT/US97/01019 and PCT/US97/01048, both of which are hereby incorporated by reference. In a preferred embodiment, the expression vector comprises the components described above and a gene encoding a variant TNF-α protein. As will be appreciated by those in the art, all combinations are possible and accordingly, as used herein, the combination of components, comprised by one or more vectors, which may be retroviral or not, is referred to herein as a “vector composition”.

A number of viral based vectors have been used for gene delivery. See for example U.S. Pat. No. 5,576,201, which is expressly incorporated herein by reference. For example, retroviral systems are known and generally employ packaging lines which have an integrated defective provirus (the “helper”) that expresses all of the genes of the virus but cannot package its own genome due to a deletion of the packaging signal, known as the psi sequence. Thus, the cell line produces empty viral shells. Producer lines can be derived from the packaging lines which, in addition to the helper, contain a viral vector, which includes sequences required in cis for replication and packaging of the virus, known as the long terminal repeats (LTRs). The gene of interest can be inserted in the vector and packaged in the viral shells synthesized by the retroviral helper. The recombinant virus can then be isolated and delivered to a subject. (See, e.g., U.S. Pat. No. 5,219,740.) Representative retroviral vectors include but are not limited to vectors such as the LHL, N2, LNSAL, LSHL and LHL2 vectors described in e.g., U.S. Pat. No. 5,219,740, incorporated herein by reference in its entirety, as well as derivatives of these vectors. Retroviral vectors can be constructed using techniques well known in the art. See, e.g., U.S. Pat. No. 5,219,740; Mann et al. (1983) Cell 33:153-159.

Adenovirus based systems have been developed for gene delivery and are suitable for delivery according to the methods described herein. Human adenoviruses are double-stranded DNA viruses that enter cells by receptor-mediated endocytosis. These viruses are particularly well suited for gene transfer because they are easy to grow and manipulate and they exhibit a broad host range in vivo and in vitro.

Adenoviruses infect quiescent as well as replicating target cells. Unlike retroviruses which integrate into the host genome, adenoviruses persist extrachromosomally thus minimizing the risks associated with insertional mutagenesis. The virus is easily produced at high titers and is stable so that it can be purified and stored. Even in the replication-competent form, adenoviruses cause only low-level morbidity and are not associated with human malignancies. Accordingly, adenovirus vectors have been developed which make use of these advantages. For a description of adenovirus vectors and their uses see, e.g., Haj-Ahmad and Graham (1986) J. Virol. 57:267-274; Bett et al. (1993) J. Virol. 67:5911-5921; Mittereder et al. (1994) Human Gene Therapy 5:717-729; Seth et al. (1994) J. Virol. 68:933-940; Barr et al. (1994) Gene Therapy 1:51-58; Berkner, K. L. (1988) BioTechniques 6:616-629; Rich et al. (1993) Human Gene Therapy 4:461-476.

In a preferred embodiment, the viral vectors used in the subject methods are AAV vectors. By an “AAV vector” is meant a vector derived from an adeno-associated virus serotype, including without limitation, AAV-1, AAV-2, AAV-3, AAV-4, AAV-5, AAVX7, etc. Typical AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. An AAV vector includes at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. For more on various AAV serotypes, see for example Cearley et al., Molecular Therapy, 16:1710-1718, 2008, which is expressly incorporated herein in its entirety by reference.

AAV expression vectors may be constructed using known techniques to provide as operatively linked components in the direction of transcription, control elements including a transcriptional initiation region, the DNA of interest and a transcriptional termination region. The control elements are selected to be functional in a thalamic and/or cortical neuron. Additional control elements may be included. The resulting construct, which contains the operatively linked components is bounded (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, e.g., Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAVX7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

Suitable DNA molecules for use in AAV vectors will include, for example, a gene that encodes a protein that is defective or missing from a recipient subject or a gene that encodes a protein having a desired biological or therapeutic effect (e.g., an enzyme, or a neurotrophic factor). The artisan of reasonable skill will be able to determine which factor is appropriate based on the neurological disorder being treated.

The selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available.

Once made, the TNF-α protein may be covalently modified. For instance, a preferred type of covalent modification of variant TNF-α comprises linking the variant TNF-α polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337, incorporated by reference. These nonproteinaceous polymers may also be used to enhance the variant TNF-α's ability to disrupt receptor binding, and/or in vivo stability. In another preferred embodiment, cysteines are designed into variant or wild type TNF-α in order to incorporate (a) labeling sites for characterization and (b) incorporate PEGylation sites. For example, labels that may be used are well known in the art and include but are not limited to biotin, tag and fluorescent labels (e.g. fluorescein). These labels may be used in various assays as are also well known in the art to achieve characterization. A variety of coupling chemistries may be used to achieve PEGylation, as is well known in the art. Examples include but are not limited to, the technologies of Shearwater and Enzon, which allow modification at primary amines, including but not limited to, lysine groups and the N-terminus See, Kinstler et al, Advanced Drug Deliveries Reviews, 54, 477-485 (2002) and M J Roberts et al, Advanced Drug Delivery Reviews, 54, 459-476 (2002), both hereby incorporated by reference.

In one preferred embodiment, the optimal chemical modification sites are 21, 23, 31 and 45, taken alone or in any combination. In an even more preferred embodiment, a TNF-α variant of the present invention includes the R31C mutation.

In a preferred embodiment, the variant TNF-α protein is purified or isolated after expression. Variant TNF-α proteins may be isolated or purified in a variety of ways known to those skilled in the art depending on what other components are present in the sample.

In another preferred embodiment, the TNF-α protein is administered via gene modified autologous or allogeneic cellular therapy, wherein the gene therapy comprises mesenchymal stem cells expressing a construct of the TNF-α protein, preferably a DN-TNF-α protein, more preferably XPRO1595.

### Trastuzumab-Resistance

As disclosed herein, when administered peripherally, selective inhibitors of solTNF, specifically DN-TNF-α proteins, and more specifically XPRO1595, may reduce or prevent MUC4 expression on HER2-positive breast cancer cells, ameliorating trastuzumab binding interferences caused by MUC4 expression, and thus, may be used to treat trastuzumab-resistant, or other anti-HER2 treatment-resistive cells in a patient having HER2-positive breast cancer.

### Treatment Methods

The terms “treatment”, “treating”, and the like, as used herein include amelioration or elimination of a disease or condition once it has been established or alleviation of the characteristic symptoms of such disease or condition. A method as disclosed herein may also be used to, depending on the condition of the patient, prevent the onset of a disease or condition or of symptoms associated with a disease or condition, including reducing the severity of a disease or condition or symptoms associated there with prior to affliction with said disease or condition. Such prevention or reduction prior to affliction refers to administration of the compound or composition as described herein to a patient that is not at the time of administration afflicted with the disease or condition. “Preventing” also encompasses preventing the recurrence or relapse-prevention of a disease or condition or of symptoms associated therewith, for instance after a period of improvement.

In one embodiment, a selective inhibitor of solTNF as described herein is administered peripherally to a patient in need thereof to reduce inflammation and/or reduce MUC4 expression in HER2-positive breast cancer cells. Peripheral administration of a selective inhibitor of solTNF as described herein can also ameliorate the effects of chemotherapy, which is often administered in conjunction with an anti-HER2 therapeutic agent, such as trastuzumab or the combination of trastuzumab and pertuzumab.

The TNF-α protein should be used in combination with one or more anti-HER2 therapeutics in patients with MUC4-positive and HER2 positive breast cancer. Combination therapy can start at the beginning of trastuzumab therapy, during trastuzumab therapy, or at any time that tumor begins to express MUC4.

Any anti-HER2 therapeutic agent known to one having skill in the art and which is shown to have improved results when used in combination with a TNF-α blockade may be similarly implemented.

### Determining MUC4 Expression

MUC4 can be measured using conventional immunohistochemistry (IHC) techniques as-appreciated by one having skill in the art (See Workman H C et al. Breast Cancer Res 2009; 11:R70, which is hereby incorporated by reference). IHC assays generally include staining one or more tissue specimens with a biomarker stain, and evaluating the stained specimen under microscope to assess an IHC score. The IHC score is selected to be one of: 0, 1+, 2+, or 3+. A score of 0 will correlate with no visible staining representations of MUC4. A score of 1+ will correlate with minor staining. A score of 2+ will correlate with borderline overexpression of MUC4 in the stained specimen. A score of 3+ will correlate with clear overexpression of MUC4 in the stained specimen.

The term “overexpressing MUC4” refers to those cancerous cells wherein MUC4 are expressed in an elevated level compared to the level in normal cells.

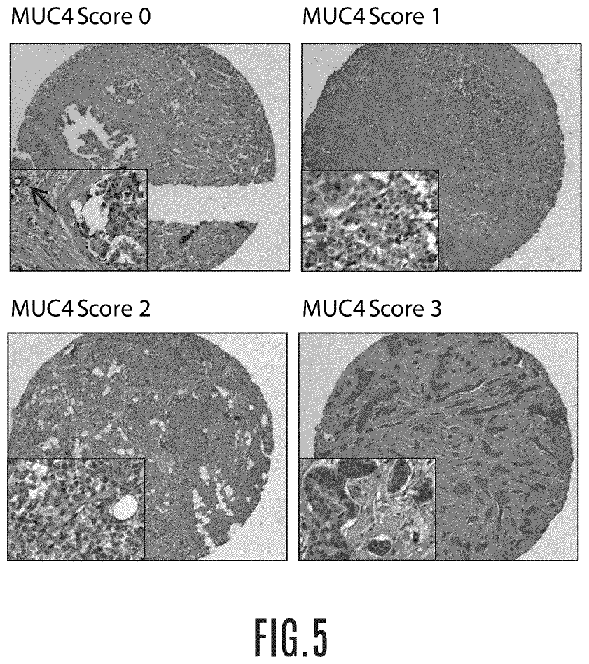

FIG. 5 shows examples of pathology analysis and scoring of MUC4 using immunohistochemistry techniques, wherein a first example shows a score of 0; a second example shows a score of 1+; a third example shows a score of 2+; and a fourth example shows a score of 3+.

In an embodiment of the invention, an inhibitor of TNF-α, more preferably a selective inhibitor of solTNF, specifically DN-TNF-α proteins, and more specifically XPRO1595, is administered as part of a combination therapy in conjunction with an anti-HER2 therapeutic agent, preferably trastuzumab, alone or in combination with pertuzumab and/or chemotherapy, if it is determined that the patient's cancerous cells are overexpressing MUC4. In this regard, MUC4 may be blocking the binding of trastuzumab, and so the treatment includes reducing MUC4 expression by way of a TNF-α blockade, thereby converting trastuzumab-resistant cells to treatable cells, and effectuating treatment with trastuzumab.

Alternatively, while determining MUC4 expression is commonly achieved using IHC assays, it is contemplated herein and within the scope of the invention to achieve the determining MUC4 expression (or overexpression) using other (non-IHC) techniques. For example, it is contemplated that MUC4 can be detected in the peripheral blood using a Surface-Enhanced Raman Scattering (SERS)-based immunoassay as would be appreciated by one with skill in the art. For example, and not limitation, each of a gold capture substrate and a gold nanoparticle may be coated with a MUC4 antibody, for example 8G7 antibody, or a surface adhesion manipulator such as 4-nitrobenzenethiol and the MUC4 antibody, and configured to bind to MUC4 in a SERS-based immunoassay, whereby MUC4 is quantitatively measure in a peripheral blood sample. In this regard, the determining of MUC4 may be quantitative, and a quantitative threshold may be pre-determined based on data from a sufficient set of normal vs. positive MUC4 overexpressing patients, as opposed to the qualitative scoring by a pathologist using IHC assays.

### Formulations

Depending upon the manner of introduction, the pharmaceutical composition may be formulated in a variety of ways. The concentration of the therapeutically active variant TNF-α protein in the formulation may vary from about 0.1 to 100 weight %. In another preferred embodiment, the concentration of the variant TNF-α protein is in the range of 0.003 to 1.0 molar, with dosages from 0.03, 0.05, 0.1, 0.2, and 0.3 millimoles per kilogram of body weight being preferred.

The pharmaceutical compositions for use in embodiments of the present invention comprise a variant TNF-α protein in a form suitable for administration to a patient. In the preferred embodiment, the pharmaceutical compositions are in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, hereby incorporated by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be.

In one embodiment provided herein, antibodies, including but not limited to monoclonal and polyclonal antibodies, are raised against variant TNF-α proteins using methods known in the art. In a preferred embodiment, these anti-variant TNF-α antibodies are used for immunotherapy. Thus, methods of immunotherapy are provided. By “immunotherapy” is meant treatment of TNF-α related disorders with an antibody raised against a variant TNF-α protein. As used herein, immunotherapy can be passive or active. Passive immunotherapy, as defined herein, is the passive transfer of antibody to a recipient (patient). Active immunization is the induction of antibody and/or T-cell responses in a recipient (patient). Induction of an immune response can be the consequence of providing the recipient with a variant TNF-α protein antigen to which antibodies are raised. As appreciated by one of ordinary skill in the art, the variant TNF-α protein antigen may be provided by injecting a variant TNF-α polypeptide against which antibodies are desired to be raised into a recipient, or contacting the recipient with a variant TNF-α protein encoding nucleic acid, capable of expressing the variant TNF-α protein antigen, under conditions for expression of the variant TNF-α protein antigen.

In another preferred embodiment, a therapeutic compound is conjugated to an antibody, preferably an anti-variant TNF-α protein antibody. The therapeutic compound may be a cytotoxic agent. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against cell cycle proteins, or binding of a radionuclide to a chelating agent that has been covalently attached to the antibody.

In a preferred embodiment, variant TNF-α proteins are administered as therapeutic agents, and can be formulated as outlined above. Similarly, variant TNF-α genes (including both the full-length sequence, partial sequences, or regulatory sequences of the variant TNF-α coding regions) may be administered in gene therapy applications, as is known in the art. These variant TNF-α genes can include antisense applications, either as gene therapy (i.e. for incorporation into the genome) or as antisense compositions, as will be appreciated by those in the art.

In a preferred embodiment, the nucleic acid encoding the variant TNF-α proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. U.S.A. 83:4143-4146 (1986), incorporated by reference). The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993), incorporated by reference). In some situations, it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), both incorporated by reference. For review of gene marking and gene therapy protocols see Anderson et al., Science 256:808-813 (1992), incorporated by reference.

In a preferred embodiment, variant TNF-α genes are administered as DNA vaccines, either single genes or combinations of variant TNF-α genes. Naked DNA vaccines are generally known in the art. Brower, Nature Biotechnology, 16:1304-1305 (1998). Methods for the use of genes as DNA vaccines are well known to one of ordinary skill in the art, and include placing a variant TNF-α gene or portion of a variant TNF-α gene under the control of a promoter for expression in a patient in need of treatment. The variant TNF-α gene used for DNA vaccines can encode full-length variant TNF-α proteins, but more preferably encodes portions of the variant TNF-α proteins including peptides derived from the variant TNF-α protein. In a preferred embodiment, a patient is immunized with a DNA vaccine comprising a plurality of nucleotide sequences derived from a variant TNF-α gene. Similarly, it is possible to immunize a patient with a plurality of variant TNF-α genes or portions thereof as defined herein. Without being bound by theory, expression of the polypeptide encoded by the DNA vaccine, cytotoxic T-cells, helper T-cells and antibodies are induced, which recognize and destroy or eliminate cells expressing TNF-α proteins.

In a preferred embodiment, the DNA vaccines include a gene encoding an adjuvant molecule with the DNA vaccine. Such adjuvant molecules include cytokines that increase the immunogenic response to the variant TNF-α polypeptide encoded by the DNA vaccine. Additional or alternative adjuvants are known to those of ordinary skill in the art and find use in the invention.

Pharmaceutical compositions are contemplated wherein a TNF-α variant of the present invention and one or more therapeutically active agents are formulated. Formulations of the present invention are prepared for storage by mixing TNF-α variant having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions. Lyophilization is well known in the art, see, e.g., U.S. Pat. No. 5,215,743, incorporated entirely by reference. Acceptable carriers, excipients, or stabilizers are nontoxic to recipients at the dosages and concentrations employed, and include buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN®, PLURONICS® or polyethylene glycol (PEG). In a preferred embodiment, the pharmaceutical composition that comprises the TNF-α variant of the present invention may be in a water-soluble form. The TNF-α variant may be present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferably sterile. This is readily accomplished by filtration through sterile filtration membranes or other methods.

### Controlled Release

In addition, any of a number of delivery systems are known in the art and may be used to administer TNF-α variants in accordance with embodiments of the present invention. Examples include, but are not limited to, encapsulation in liposomes, microparticles, microspheres (e.g. PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT®, and poly-D-(−)-3-hydroxyburyric acid. It is also possible to administer a nucleic acid encoding the TNF-α of the current invention, for example by retroviral infection, direct injection, or coating with lipids, cell surface receptors, or other transfection agents. In all cases, controlled release systems may be used to release the TNF-α at or close to the desired location of action.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, incorporated entirely by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be. The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. In a further embodiment, the variant TNF-α proteins are added in a micellular formulation; see U.S. Pat. No. 5,833,948, incorporated entirely by reference. Alternatively, liposomes may be employed with the TNF-α proteins to effectively deliver the protein. Combinations of pharmaceutical compositions may be administered. Moreover, the TNF-α compositions of the present invention may be administered in combination with other therapeutics, either substantially simultaneously or co-administered, or serially, as the need may be.

Dosage forms for the topical or transdermal administration of a DN-TNF-protein disclosed herein include powders, sprays, ointments, pastes, creams, lotions, gels, solutions, patches and inhalants. The DN-TNF-protein may be mixed under sterile conditions with a pharmaceutically-acceptable carrier, and with any preservatives, buffers, or propellants which may be required. Powders and sprays can contain, in addition to the DN-TNF-protein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

### Methods of Administration

The administration of the selective inhibitor of solTNFin accordance with embodiments of the present invention, preferably in the form of a sterile aqueous solution, is done peripherally, in a variety of ways, including, but not limited to, orally, subcutaneously, intravenously, intranasally, transdermally, intraperitoneally, intramuscularly, intrapulmonary, vaginally, rectally, or intraocularly. In some instances, the selective inhibitor of solTNF may be directly applied as a solution, salve, cream or spray. The selective inhibitor of solTNF may also be delivered by bacterial or fungal expression into the human system (e.g., WO 04046346 A2, hereby incorporated by reference).

Subcutaneous

Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate. A selective inhibitor of solTNF may be more amenable to subcutaneous administration due to, for example, increased potency, improved serum half-life, or enhanced solubility.

Intravenous

As is known in the art, protein therapeutics are often delivered by IV infusion or bolus. The selective inhibitor of solTNF may also be delivered using such methods. For example, administration may be by intravenous infusion with 0.9% sodium chloride as an infusion vehicle.

Inhaled

Pulmonary delivery may be accomplished using an inhaler or nebulizer and a formulation comprising an aerosolizing agent. For example, inhalable technology, or a pulmonary delivery system may be used. The selective inhibitor of solTNF may be more amenable to intrapulmonary delivery. The selective inhibitor of solTNF may also be more amenable to intrapulmonary administration due to, for example, improved solubility or altered isoelectric point.

Oral Delivery

Furthermore, the selective inhibitor of solTNF may be more amenable to oral delivery due to, for example, improved stability at gastric pH and increased resistance to proteolysis.

Transdermal

Transdermal patches may have the added advantage of providing controlled delivery of the selective inhibitor of solTNF to the body. Dissolving or dispersing DN-TNF-protein in the proper medium can make such dosage forms. Absorption enhancers can also be used to increase the flux of DN-TNF-protein across the skin. Either providing a rate controlling membrane or dispersing DN-TNF-protein in a polymer matrix or gel can control the rate of such flux.

Intraocular

Ophthalmic formulations, eye ointments, powders, solutions and the like, are also contemplated as being suitable for use in embodiments of this invention.

In a preferred embodiment, the selective inhibitor of solTNF is administered as a therapeutic agent, and can be formulated as outlined above. Similarly, variant TNF-α genes (including both the full-length sequence, partial sequences, or regulatory sequences of the variant TNF-α coding regions) may be administered in gene therapy applications, as is known in the art. These variant TNF-α genes can include antisense applications, either as gene therapy (i.e. for incorporation into the genome) or as antisense compositions, as will be appreciated by those in the art.

In a preferred embodiment, the nucleic acid encoding the variant TNF-α proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. (Zamecnik et al., Proc. Natl. Acad. Sci. U.S.A. 83:4143-4146 (1986), incorporated entirely by reference). The oligonucleotides can be modified to enhance their uptake, e.g. by substituting their negatively charged phosphodiester groups by uncharged groups.

Dosage

Dosage may be determined depending on the complication being treated and mechanism of delivery. Typically, an effective amount of the selective inhibitor of solTNF, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 2000 mg per kilogram body weight per day. An exemplary treatment regime entails administration once every day or once a week or once a month. A DN-TNF protein may be administered on multiple occasions. Intervals between single dosages can be daily, weekly, monthly or yearly. Alternatively, A DN-TNF protein may be administered as a sustained release formulation, in which case less frequent administration is required. Dosage and frequency vary depending on the half-life of the agent in the subject. The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the patent can be administered a prophylactic regime.

Toxicity

Suitably, an effective amount (e.g., dose) of a DN-TNF protein described herein will provide therapeutic benefit without causing substantial toxicity to the subject. Toxicity of the agent described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in human. The dosage of the agent described herein lies suitably within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

### Invasive Micropapillary Carcinoma

Invasive micropapillary carcinoma of the breast (IMPC) is a histological tumor variant that occurs with low frequency characterized by an inside-out formation of tumor clusters with a pseudopapillary arrangement. IMPC is an aggressive tumor with poor clinical outcome. In addition, this histological subtype usually expresses human epidermal growth factor receptor 2 (HER2) which also correlates with a more aggressive tumor. The clinical significance of IMPC in HER2-positive breast cancer patients treated with adjuvant trastuzumab was studied (See Mercogliano et al. BMC Cancer (2017) 17:895, the entire contents of which are hereby incorporated by reference). Mucin 4 (MUC4) expression was analyzed as a novel biomarker to identify IMPC.

Eighty-six HER2-positive breast cancer patients treated with trastuzumab and chemotherapy in the adjuvant setting were retrospectively studied. The association of the IMPC component with clinicopathological parameters at diagnosis and its prognostic value was explored. MUC4 expression in IMPC was compared with respect to other histological breast cancer subtypes by immunohistochemistry.

IMPC, either as a pure entity or associated with invasive ductal carcinoma (IDC), was present in 18.6% of HER2-positive cases. It was positively correlated with estrogen receptor expression and tumor size and inversely correlated with patient's age. Disease-free survival was significantly lower in patients with IMPC (hazard ratio=2.6; 95%, confidence interval 1.1-6.1, P=0.0340). MUC4, a glycoprotein associated with metastasis, was strongly expressed in all IMPC cases tested. IMPC appeared as the histological breast cancer subtype with the highest MUC4 expression compared to IDC, lobular and mucinous carcinoma.

In HER2-positive breast cancer, the presence of IMPC should be carefully examined. As it is often not informed, because it is relatively difficult to identify or altogether overlooked, it is proposed that MUC4 expression is a useful biomarker to highlight IMPC presence. Patients with MUC4-positive tumors with IMPC component should be more frequently monitored and/or receive additional therapies.

In particular, it is proposed that patients with MUC4-positive tumors with IMPC component should be treated with a TNF-α blockade, and more specifically, an inhibitor of solTNF as described herein.

### CONCLUSION

A method is disclosed for treating a patient suffering from HER2-positive breast cancer, the method comprising: (i) determining mucin-4 (MUC4) expression in cells of the patient; and (ii) if the MUC4 expression is greater than a predetermined-threshold, administering to the patient a therapeutically effective amount of a selective inhibitor of soluble tumor necrosis factor (solTNF) in combination with an anti-HER2 therapeutic agent, whereby said patient is treated.

In a preferred embodiment, said determining the MUC4 expression comprises: obtaining an immunohistochemistry (IHC) score between 0 and 3+. In this regard, the predetermined-threshold is 2+, correlating with at least borderline overexpression of MUC4.

In another embodiment, a quantitative technique, such as a SERS-based immunoassay may be used to determine the MUC4 expression in the patient and basing treatment decisions as described herein.

In some embodiments, the selective inhibitor of solTNF comprises: a dominant negative tumor necrosis factor (DN-TNF)-α protein, a nucleic acid encoding the DN-TNF-α protein, or a combination thereof. In a preferred embodiment, the DN-TNF-α protein is XPRO1595.

In an embodiment, the method comprises administering XPRO1595 in a dose between 0.1 mg/kg and 10.0 mg/kg.

The DN-TNF-α protein is preferably administered: intravenously; subcutaneously; orally; via aerosol; via topical application; or via gene therapy.

In an embodiment, the DN-TNF-α protein can be administered via gene modified autologous or allogeneic cellular therapy. The gene therapy may comprise use of mesenchymal stem cells expressing a construct of the DN-TNF-α protein.

In a preferred embodiment, the anti-HER2 therapeutic agent comprises trastuzumab. In other embodiments, the anti-HER2 therapeutic agent further comprises pertuzumab, chemotherapy, or a combination thereof.

### Experimental Investigations

As set forth above, HER2 positive is a subtype that affects 13-20% of breast cancer (BC) patients. They receive trastuzumab (T), an anti-HER2 monoclonal antibody, but resistance events hamper its clinical benefit in 40-60% of the cases.

It was demonstrated that TNFα overexpression turned T-sensitive cells and tumors into resistant ones by upregulating MUC4 expression (See Maria F. Mercogliano et. al., Clin Cancer Res; 23(3), 636-648, the entire contents of which are hereby incorporated by reference). Pertuzumab (P, a monoclonal antibody that disrupts HER2/HER3 dimerization) is another anti-HER2 therapy that is used in combination with T.

It has been demonstrated that MUC4 expression, in HER2-positive breast cancer samples, is an independent predictor of poor disease-free survival in patients treated with adjuvant trastuzumab.

A strategy of implementing a TNFα blockade was employed, either with Etanercept (E) or the dominant negative protein XPRO1595 (DN) in JIMT-1, de novo T and T+P resistant cell line which produces TNFα. JIMT-1 tumors were established in female nude mice to explore whether TNFα blockade overcomes T+P resistance. Animals were treated with 5 mg/kg of IgG, P, T+P, 10 mg/kg of DN or P+T+DN i.p. twice a week. The combination of T+P+DN inhibited tumor growth vs. T+P or P+DN (p<0.05). In addition, the administration of DN induces MUC4 expression downregulation in JIMT-1 tumors. Proliferation of cells treated with IgG, T, P, DN, E (10 μg/ml, 10 μg/ml, 10 μg/ml, 2 μg/ml and 5 μg/ml respectively) as monotherapy and in different combinations was evaluated by cell count or [3H]-thymidine incorporation. The combination of TNFα blockade with T+P inhibited cell proliferation vs. IgG, P+T, P+E, P+DN (p<0.0001).

While treatment-resistance MUC4+HER2+ breast cancer has been shown to become treatable upon the administration of a TNF blockade, for example, using DN-TNF technology, it is further contemplated that other MUC4+HER2+ cancers, including: inter alia, gastric, pancreatic, and liver cancer, can be treated in a similar manner. This is because MUC4 creates a physical barrier to HER2 binding, or steric hinderance, which prevents anti-HER2 therapy from working. By down-regulating MUC4 by way of TNF blockade as described herein, this enables treatment with anti-HER2 therapy, such as trastuzumab, in otherwise treatment-resistive cancer. Thus, the invention is not limited to treatment of MUC4+HER2+ breast cancer, but may be reasonably expanded to accomplish the treatment of any and all MUC4+HER2+ cancers, whether such MUC4+HER2+ cancers are known or later discovered.

Also, as provided in the below examples, it was discovered that DNTNF technology, such as administration of XPRO1595, can be used to convert a cold tumor to a hot tumor for purposes of treatment.

### EXAMPLES

### Example 1: Cell Proliferation in JIMT-1 Cell Line Evaluated By ³[H]-Thymidine Incorporation Assay at 48 h of Treatment with 18 h Pulse

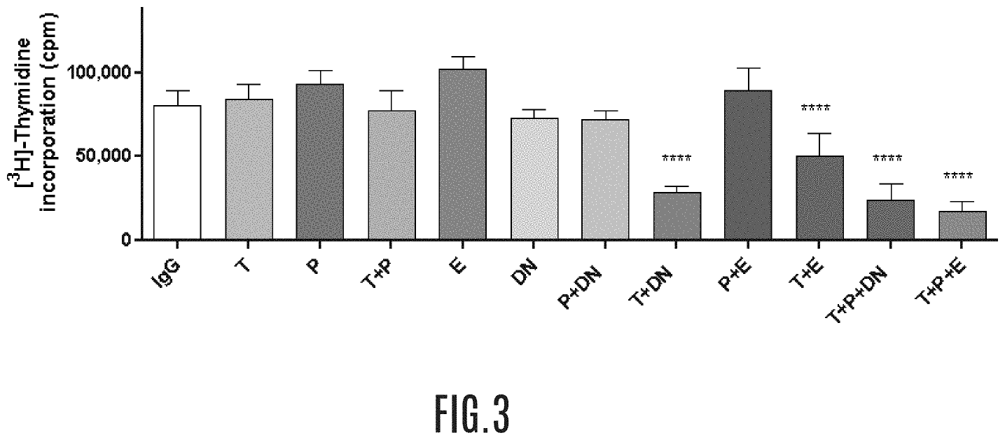

Cell proliferation in JIMT-1 cell line was evaluated by ³[H]-thymidine incorporation assay at 48 h of treatment with 18 h pulse (See Rivas M A, et al., Exp Cell Res 2008; 314:509-29, the contents of which are hereby incorporated by reference). Cells were treated with Immunoglobulin G (IgG), trastuzumab (T; 10 μg/ml), pertuzumab (P; 10 μg/ml), etanercept (E; 5 μg/ml), or XPRO1595 (DN; 2 μg/ml), alone or in different combinations. Data was analyzed by one-way ANOVA coupled with a Tukey's post hoc test. The data as shown in FIG. 3 represents mean±standard deviation *p<0.05, **p<0.01, ***p<0.001 vs. IgG.

These results as shown in FIG. 3 indicate cell proliferation is significantly reduced in vitro by combination trastuzumab and XPRO1595 compared with other experimental iterations. The combination trastuzumab plus pertuzumab and XPRO1595 achieved similar results; however, the data indicates that pertuzumab had negligible, if any, impact.

### Example 2: JIMT Rodent Study

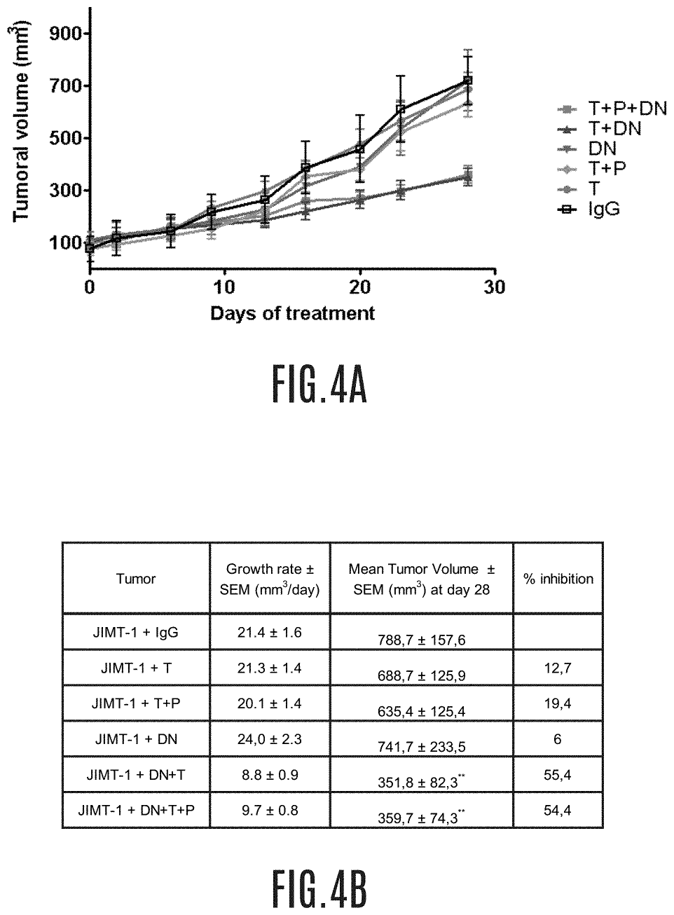

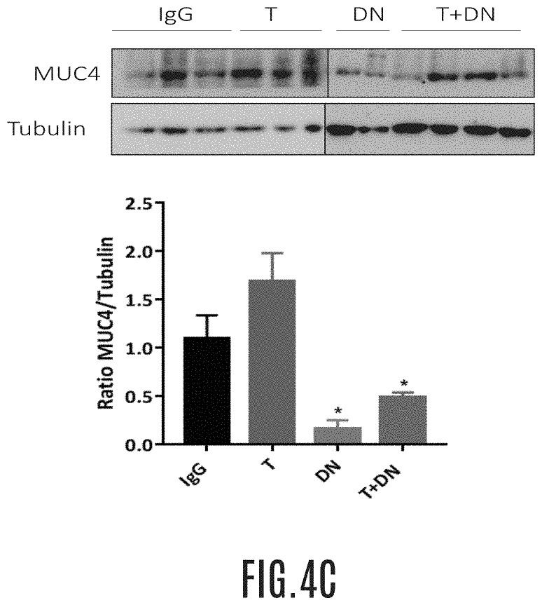

Female nude mice were injected with 3×10⁶ JIMT-1 cells. After the tumors were established, animals were treated intraperitoneally (ip) twice a week with Immunoglobulin G (IgG; 5 mg/kg), trastuzumab (T; 5 mg/kg), XPRO1595 (DN; 10 mg/kg), T+P (5 mg/kg each), T+DN (5 mg/kg each), or T+P+DN(5 mg/kg each). Tumor growth was measured during 28 days as shown in FIG. 4A, and growth rate was calculated at the end of the experiment as shown in FIG. 4B. ** p<0.01 vs. IgG treated. FIG. 4C shows protein extracts obtained from tumors subjected to different treatments as was described in FIG. 4A. MUC4 expression was determined by western blot (upper panel). Quantification of MUC4 expression is shown as a ratio using tubulin as a housekeeping protein (lower panel). * p<0.05 vs. IgG treated. The results confirm significant improvement in the treatment of JIMT-1 bearing mice with combination trastuzumab plus XPRO1595 (with and without pertuzumab). Also demonstrated, XPRO1595 is able to downregulate MUC4 expression in JIMT-1 tumor in vivo.

### Example 3: Scoring MUC4 Using Immunohistochemistry Analysis

Tissue microarrays were used to establish a score of MUC4 by IHC analysis as described by Workman et al. Score 0 represents no stain to less than 30% of cells stained faintly, 1+ greater than 30% of cells stained with light to moderate intensity, 2+ greater than 50% of cells stained moderately, 3+ intense staining of majority of the epithelial population. The arrow in the inset of MUC4 (score 0) shows positive staining in endothelial cells, used as an internal control. These results are shown in FIG. 5.

### Example 4: MUC4 Expression is Associated with Poor Outcome in Patients Treated with Adjuvant Trastuzumab

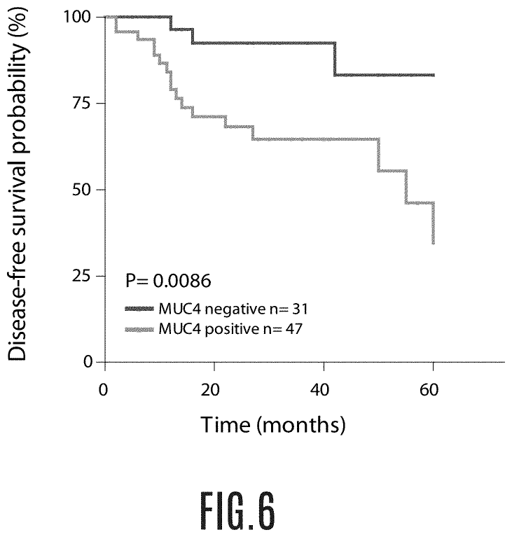

FIG. 6 shows Kaplan-Meier analysis of the probability of DFS of a cohort of 78 patients with HER2-positive tumors, who received adjuvant trastuzumab treatment, based on the expression of MUC4. These results demonstrate that MUC4 expression is associated with poor outcome in patients treated with adjuvant trastuzumab

### Example 5: MUC4 is an Independent Prognostic Biomarker of Poor Outcome in Patients Treated with Adjuvant Trastuzumab

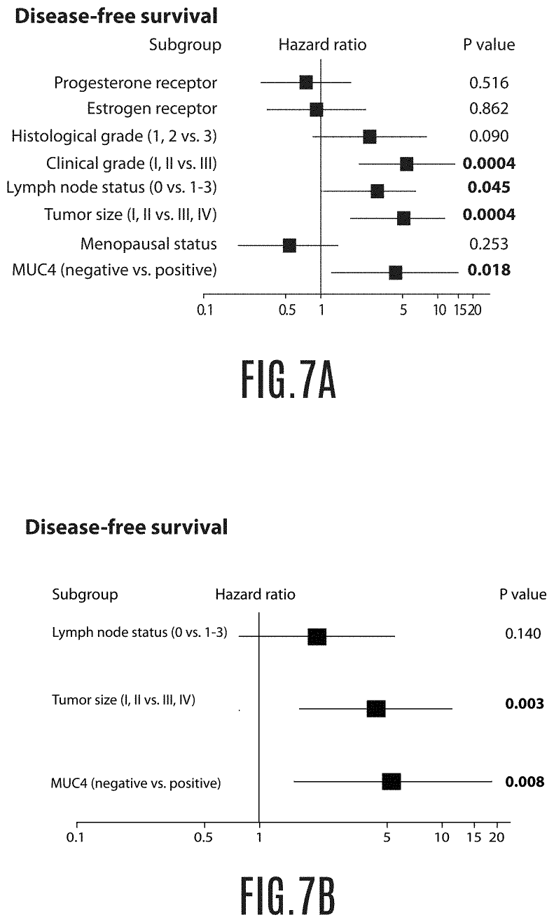

FIG. 7A shows COX univariate analysis, and FIG. 7B shows multivariate analysis, of disease fee survival (DFS) for a number of subgroups as labeled in the illustrations. These results demonstrate that MUC4 is an independent prognostic biomarker of poor outcome in patients treated with adjuvant trastuzumab.

### Example 6: Soluble TNFα Blockade Decreases Migration and Invasion in Trastuzumab-Resistant HER2+ Breast Cancer Cells

The course of tumor metastasis entails a series of steps that includes the tumor cell capacity to migrate and invade in order to form a secondary tumor in distant organs. To study whether XPRO1595 might modify the tumor cells metastatic phenotype, migration and invasion assays using JIMT-1 cells were performed.

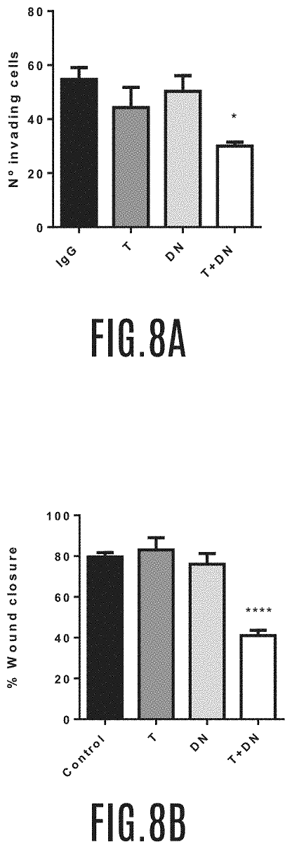

FIG. 8A shows that migration of JIMT-1 cells, performed by the scratch assay, was impaired when trastuzumab was used in combination with XPRO1595.

Similar results were obtained in the invasion assay (FIG. 8B). It is therefore surprisingly discovered that XPRO1595 is not only is able to decrease the proliferation of HER2+ trastuzumab resistant cancer cells in presence of this monoclonal antibody (FIG. 3), but also hampers the migratory and invasive activity of them.

### Example 7: MUC4 has a Key Role in TNFα-Induced Trastuzumab Resistance In Vivo

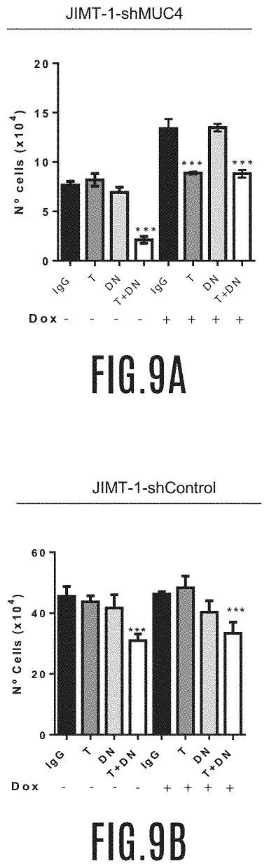

To study the participation of MUC4 on trastuzumab resistance, the trastuzumab-resistant JIMT-1 cell line was transduced with a plasmid encoding a doxycycline-inducible shRNA targeting MUC4, and JIMT-1-shMUC4 cells were obtained. Also, JIMT-1 cells were transduced with the corresponding Control shRNA (JIMT-1-shControl). It was observed that MUC4 silencing by doxycycline treatment sensitizes JIMT-1 cells to trastuzumab and that addition of XPRO1595 did not further induce a decrease in cell proliferation (FIG. 9A). JIMT-1shMUC4 without doxycycline, JIMT-1-shControl without or with doxycycline decreased their proliferation only when trastuzumab and XPRO1595 were present (FIG. 9A and FIG. 9B, respectively), in similar manner than JIMT-1 wild type (FIG. 3).

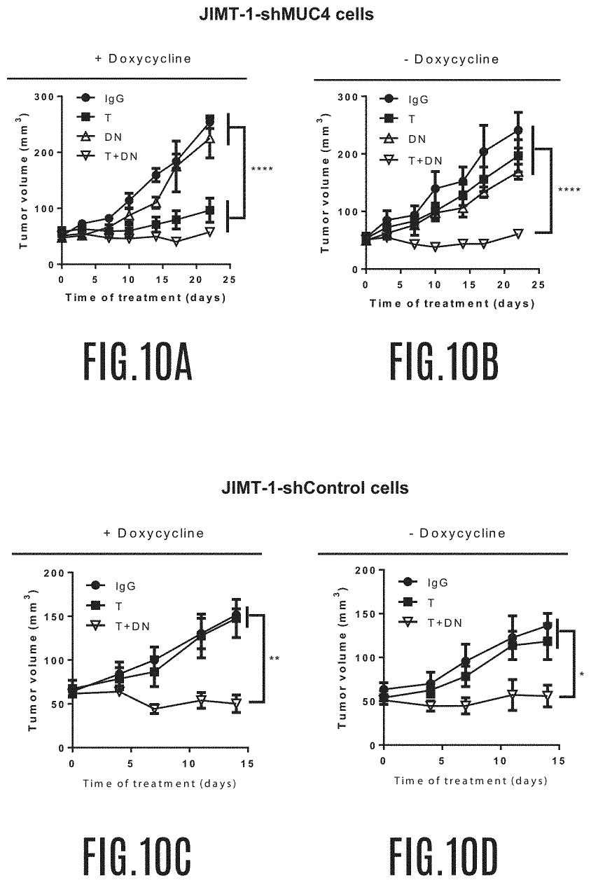

To evaluate the in vivo effect of MUC4 downregulation in trastuzumab-resistant tumors, we injected JIMT-shMUC4 cells in female nude mice. After the tumors were established, animals were randomly assigned to the experimental (+doxycycline 2 mg/ml in drinking water) or control group (−doxycycline). Both groups were treated with IgG, trastuzumab, (both 5 mg/kg), XPRO1595 (10 mg/kg), or trastuzumab+XPRO1595 i.p. twice a week and tumor volume was monitored routinely. Knockdown of MUC4 expression revealed that trastuzumab treatment was effective in inhibiting JIMT-1-shMUC4 tumor growth (62%) at comparable levels to trastuzumab+XPRO1595 administration (76% vs. IgG, FIG. 10A). In control groups, only trastuzumab+XPRO1595 administration was able to inhibit tumor growth (75%, respectively vs. IgG, FIG. 10B). JIMT-1-sh-Control tumors with or without doxycycline administration behaved similar (FIG. 10C and FIG. 10D) to JIMT-1 wildtype tumors (FIG. 4A). This is the first in vivo demonstration that MUC4 is a mediator of trastuzumab resistance. In addition, these data suggest that MUC4 is a major player in TNFα-induced trastuzumab resistance in vivo.

### Example 8: MUC4 Expression Generates an Immunosuppressive Tumor Microenvironment

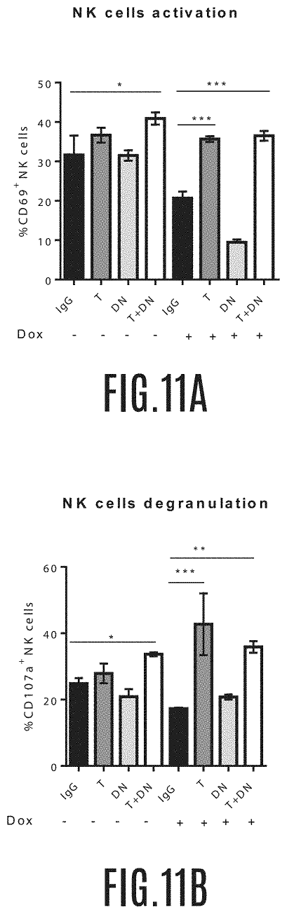

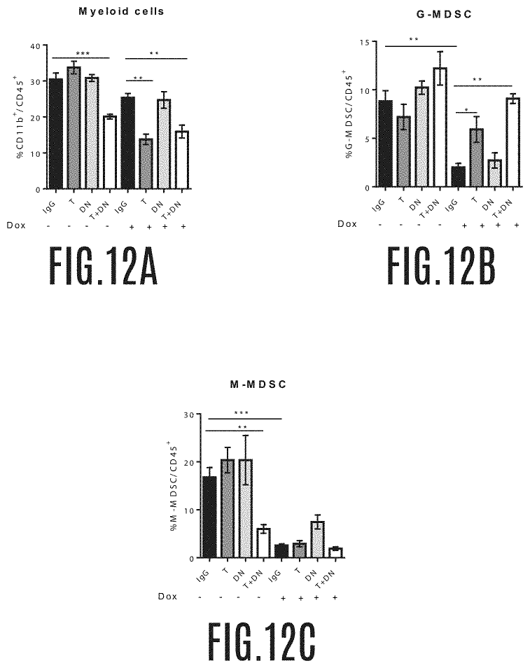

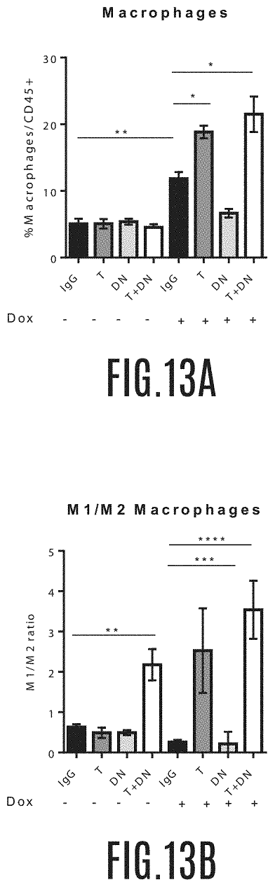

To evaluate the participation of innate immune response in the antitumor effect observed above, tumor-infiltrating immune cells were evaluated by immunofluorescence and analyzed by flow cytometry. It was discovered that tumor growth inhibition was accompanied by an increase in NK cells activation and degranulation (FIG. 11A and FIG. 11B, respectively) and a decrease in the recruitment of myeloid cells vs. IgG (FIG. 12A). Interestingly, MUC4 silencing sharply decreased granulocytic-myeloid-derived suppressor cells (G-MDSC) and macrophage-MDSC (M-MDSC) presence in the tumor microenvironment when we compared IgG groups treated or not with doxycycline (FIG. 12B and FIG. 12C). Trastuzumab and trastuzumab+XPRO1595 increases G-MDSC vs IgG with doxycycline (FIG. 12B). The combination therapy of trastuzumab+XPRO1595 sharply decreases M-MDSC presence in JIMT-shMUC4 tumors without doxycycline vs IgG (FIG. 12C). Macrophages influx to the tumor bed was stimulated by the absence of MUC4 and was further increased in tumors treated with trastuzumab, or trastuzumab+XPRO1595, vs IgG (FIG. 13A). Notably, a rise in M1/M2 macrophages ratio was observed in JIMT-shMUC4 tumors without doxycycline treated with trastuzumab+XPRO1595 vs IgG group (FIG. 13B). When MUC4 was silenced, an increase in M1/M2 macrophages ratio was observed in the trastuzumab and trastuzumab+XPRO1595 treated groups vs IgG group (FIG. 13B).

Taking these data as a whole, it is concluded that MUC4 favors an immunosuppressive tumor microenvironment by avoiding macrophages influx and their differentiation to the M1 antitumor subtype, and fostering MDSC recruitment. Soluble TNFα (sTNF) blockade allows trastuzumab action and consequently an antitumor innate immune response.

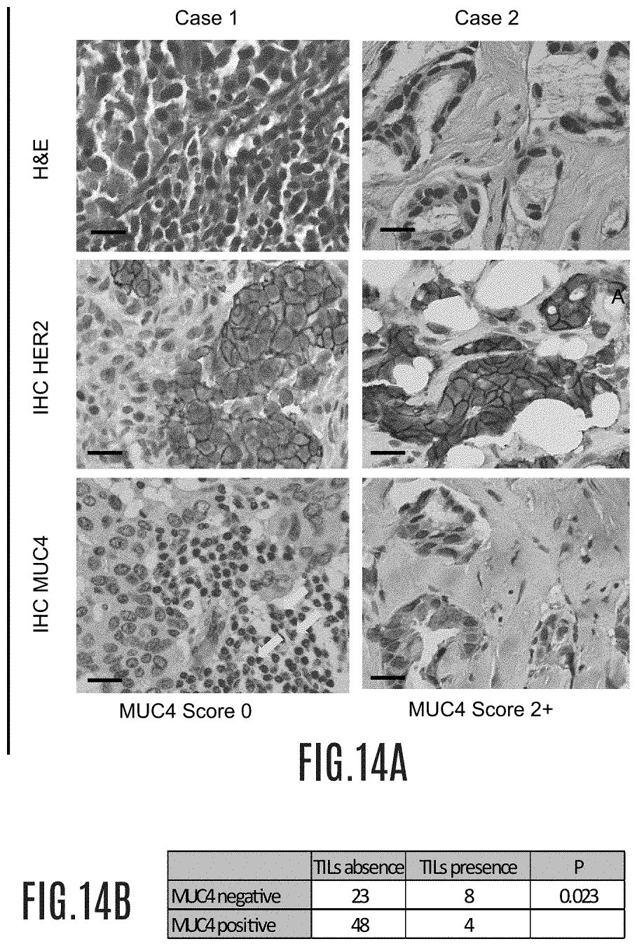

It is known that tumor infiltrating lymphocytes (TILs) presence in HER2+ breast cancer is correlated with good prognosis and success of trastuzumab treatment. Accordingly, HER2+ breast cancer samples in which MUC4 expression had already been studied were analyzed (FIG. 6). FIG. 14 shows representative cases of HER2+/MUC4+ tumors that lack TILs and HER2+/MUC4− tumors that had abundant TILs. The inverse correlation between MUC4 expression and TILs presence is shown in FIG. 14B. This finding supports the discovery that MUC4 plays an important role in tumor immune evasion.

### Findings

With the experimental results as described herein, it has been appreciated that:

treatment with TNFα-blocking agents in combination with trastuzumab plus pertuzumab resulted in a significant decrease in cell proliferation compared to IgG, the monotherapies or trastuzumab plus pertuzumab, suggesting that TNFα blockade sensitizes cells resistant to trastuzumab plus pertuzumab treatment;

the combination of trastuzumab plus pertuzumab and XPRO1595 caused a significant decrease in tumor volume. Meanwhile, tumors were unresponsive to pertuzumab plus trastuzumab, and pertuzumab plus XPRO1595 treatments;

the effect of trastuzumab plus XPRO1595 is indistinguishable from that of trastuzumab+pertuzumab and XPRO1595, indicating that pertuzumab is not contributing to the antiproliferative effect of trastuzumab plus XPRO1595;

treatment with XPRO1595 induces MUC4 downregulation in HER2-positive breast cancer in vivo;

soluble TNFα blockade decreases migration and invasion in trastuzumab-resistant HER2+ breast cancer cells;

MUC4 has a key role in TNFα-induced trastuzumab resistance in vivo; and

MUC4 expression generates an immunosuppressive tumor microenvironment.

What is claimed is:

1. A method for treating a patient suffering from HER2-positive breast cancer, the method comprising:
determining MUC4 expression in cancer cells of the patient; and
if the MUC4 expression is greater than or equal to a predetermined-threshold, administering to the patient a therapeutically effective amount of a selective inhibitor of soluble tumor necrosis factor (solTNF) in combination with an anti-HER2 therapeutic agent, whereby said patient is treated.

2. The method of claim 1, wherein said determining the MUC4 expression comprises: obtaining an immunohistochemistry (IHC) score between 0 and 3+.

3. The method of claim 2, wherein the predetermined-threshold is 2+.

4. The method of claim 1, wherein the selective inhibitor of solTNF comprises: a dominant negative tumor necrosis factor (DN-TNF)-α protein, a nucleic acid encoding the DN-TNF-α protein, or a combination thereof.

5. The method of claim 4, wherein the DN-TNF-α protein is XPRO1595.

6. The method of claim 5, wherein the method comprises administering XPRO1595 in a dose between 0.1 mg/kg and 10.0 mg/kg.

7. The method of claim 4, wherein the DN-TNF-α protein is administered: intravenously; subcutaneously; orally; via aerosol; via topical application; or via gene therapy.

8. The method of claim 4, wherein the DN-TNF-α protein is administered via gene modified autologous or allogeneic cellular therapy.

9. The method of claim 8, wherein the gene therapy comprises mesenchymal stem cells expressing a construct of the DN-TNF-α protein.

10. The method of claim 1, wherein the anti-HER2 therapeutic agent comprises trastuzumab.

11. The method of claim 10, wherein the anti-HER2 therapeutic agent further comprises pertuzumab.

12. The method of claim 1, wherein the anti-HER2 therapeutic agent is administered alone or in combination with chemotherapy.

13. The method of claim 1, wherein said administering to the patient a therapeutically effective amount of a selective inhibitor of solTNF in combination with an anti-HER2 therapeutic agent comprises one of:
a series administration of the selective inhibitor of solTNF followed by administration of the anti-HER2 therapeutic agent;
a series administration of the anti-HER2 therapeutic agent followed by administration of the selective inhibitor of solTNF;
a concurrent administration of the selective inhibitor of solTNF and the anti-HER2 therapeutic agent; or
administration of a formulation comprising the selective inhibitor of solTNF and the anti-HER2 therapeutic agent.

14. A method for treating a patient suffering from MUC4-positive, HER2-positive breast cancer, the method comprising:
administering to the patient each of:
a therapeutically effective amount of a selective inhibitor of soluble tumor necrosis factor (solTNF), and
a therapeutically effective amount of an anti-HER2 therapeutic agent;
whereby said patient is treated.

15. A method for treating a patient suffering from trastuzumab-resistant HER2-positive breast cancer, the method comprising:
determining MUC4 expression in cancer cells of the patient; and
if the MUC4 expression is greater than or equal to a predetermined-threshold, administering to the patient a therapeutically effective amount of a selective inhibitor of soluble tumor necrosis factor (solTNF) in combination with an anti-HER2 therapeutic agent, whereby said patient is treated.

16. The method of claim 15, wherein said determining the MUC4 expression comprises: obtaining an immunohistochemistry (IHC) score between 0 and 3+; and the predetermined-threshold is 2+.

17. The method of claim 15, wherein the selective inhibitor of solTNF comprises: a dominant negative tumor necrosis factor (DN-TNF)-α protein, a nucleic acid encoding the DN-TNF-α protein, or a combination thereof.

18. The method of claim 17, wherein the DN-TNF-α protein is XPRO1595.

19. The method of claim 18, wherein the method comprises administering XPRO1595 in a dose between 0.1 mg/kg and 10.0 mg/kg.

20. The method of claim 17, wherein the DN-TNF-α protein is administered: intravenously; subcutaneously; orally; via aerosol; via topical application; or via gene therapy.

21. The method of claim 15, wherein the anti-HER2 therapeutic agent comprises trastuzumab.

22. The method of claim 21, wherein the anti-HER2 therapeutic agent further comprises pertuzumab.

23. The method of claim 15, wherein the anti-HER2 therapeutic agent is administered alone or in combination with chemotherapy.

24. The method of claim 15, wherein said administering to the patient a therapeutically effective amount of a selective inhibitor of solTNF in combination with an anti-HER2 therapeutic agent comprises one of:
a series administration of the selective inhibitor of solTNF followed by administration of the anti-HER2 therapeutic agent;
a series administration of the anti-HER2 therapeutic agent followed by administration of the selective inhibitor of solTNF;
a concurrent administration of the selective inhibitor of solTNF and the anti-HER2 therapeutic agent; or
administration of a formulation comprising the selective inhibitor of solTNF and the anti-HER2 therapeutic agent.


SEQUENCE LISTING (3 sequences)

SEQ ID NO: 1 - DNA, 495 bp, Homo sapiens
atgcaccaccaccaccaccacgtacgctcctcctcccgcactccgtccgacaaaccggtagctcacgtagtagctaacccgcaggctgaaggtcagctgcagtggctgaaccgccgcgctaacgctctgctggctaacggtgtagaactgcgcgacaaccagctggtagtaccgtccgaaggtctgtacctgatctactcccaggtactgttcaaaggtcagggttgtccgtccactcacgtactgctgactcacactatctcccgcatcgctgtatcctaccagactaaagtaaacctgctgtccgctatcaaatccccgtgtcagcgcgaaactccggaaggtgctgaagctaaaccgtggtacgaaccgatctacctgggtggtgtattccagctggaaaaaggtgaccgcctgtccgctgaaatcaaccgcccggactacctggacttcgctgaatccggtcaggtatacttcggtatcatcgctctgtga

SEQ ID NO: 2 - protein, 164 aa, Homo sapiens
MHHHHHHVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 157 aa, Homo sapiens
VRSSSRTPSDKPVAHVVANPQAEGQLQWLNRRANALLANGVELRDNQLVVPSEGLYCIYSQVLFKGQGCPSTHVLLTHTISRIAVSYQTKVNLLSAIKSPCQRETPEGAEAKPWYEPITLGGVFQLEKGDRLSAEINRPDYLDFAESGQVYFGIIAL